(19) United States
(12) Patent Application Publication
(10) Pub. No.: US 2006/0142613 A1
(43) Pub. Date: Jun. 29, 2006

(54) Process for producing (meth)acrylic acid

(75) Inventors: Shuhei Yada, Mie-ken (JP); Yasushi Ogawa, Mie-ken (JP); Yoshiro Suzuki, Mie-ken (JP); Kenji Takasaki, Mie-ken (JP)

Correspondence Address: NIXON & VANDERHYE, PC, 901 NORTH GLEBE ROAD, 11TH FLOOR, ARLINGTON, VA, 22203, US

(73) Assignee: Mitsubishi Chemical Corporation, Tokyo (JP)

(21) Appl. No.: 11/359,618

(22) Filed: Feb. 23, 2006

Related U.S. Application Data
(62) Division of application No. 10/827,529, filed on Apr. 20, 2004.
Application No. 10/827,529 is a continuation of application No. PCT/JP02/13179, filed on Dec. 17, 2002.

(30) Foreign Application Priority Data
Dec. 19, 2001 (JP) 2001-386657
Jan. 17, 2002 (JP) 2002-8918

(51) Int. Cl. C07C 51/42

(52) U.S. Cl. 562/600

(57) ABSTRACT

In a process for producing (meth)acrylic acid comprising contacting a reaction gas containing (meth)acrylic acid obtained by gas-phase catalytic oxidation with an absorbent solvent to prepare a (meth)acrylic acid solution; and introducing the (meth)acrylic acid solution into a distillation column to purify (meth)acrylic acid, after a dissolved oxygen concentration in the (meth)acrylic acid solution to be introduced into the distillation column is adjusted to not less than 12 ppm by weight, the (meth)acrylic acid solution is fed to the distillation column. In addition, upon an azeotropic dehydration distillation step, a phenol-based polymerization inhibitor is fed to an azeotropic dehydration distillation column from a position not lower than a raw material feed stage thereof, and a copper-based polymerization inhibitor is fed to the azeotropic dehydration distillation column from a position lower than the raw material feed stage. According these methods, the production of polymers of (meth)acrylic acid and polymerization clogging in the distillation column are prevented, so that it is possible to stably purify (meth)acrylic acid by distillation for a long period of time.

### TECHNICAL FIELD

The present invention relates to a process for producing (meth)acrylic acid, and more particularly to a process for producing (meth)acrylic acid in which a (meth)acrylic acid solution obtained by subjecting propane, propylene or acrolein, or isobutylene or t-butyl alcohol to gas-phase catalytic oxidation reaction is purified in a distillation column while preventing the (meth)acrylic acid from being polymerized, thereby ensuring stable distillation purification of (meth)acrylic acid for a long period of time.

Meanwhile, in the present specification, the “(meth)acrylic acid” generally means acrylic acid and methacrylic acid, and may include either one or both of these acids.

### BRIEF DESCRIPTION OF THE DRAWINGS

FIG. 1 is a flow sheet showing an example of a dissolved oxygen concentration adjusting means for an aqueous acrylic acid solution which is usable in the present invention.

FIGS. 2 and 3 are flow sheets showing a process for producing acrylic acid.

### BACKGROUND ARTS

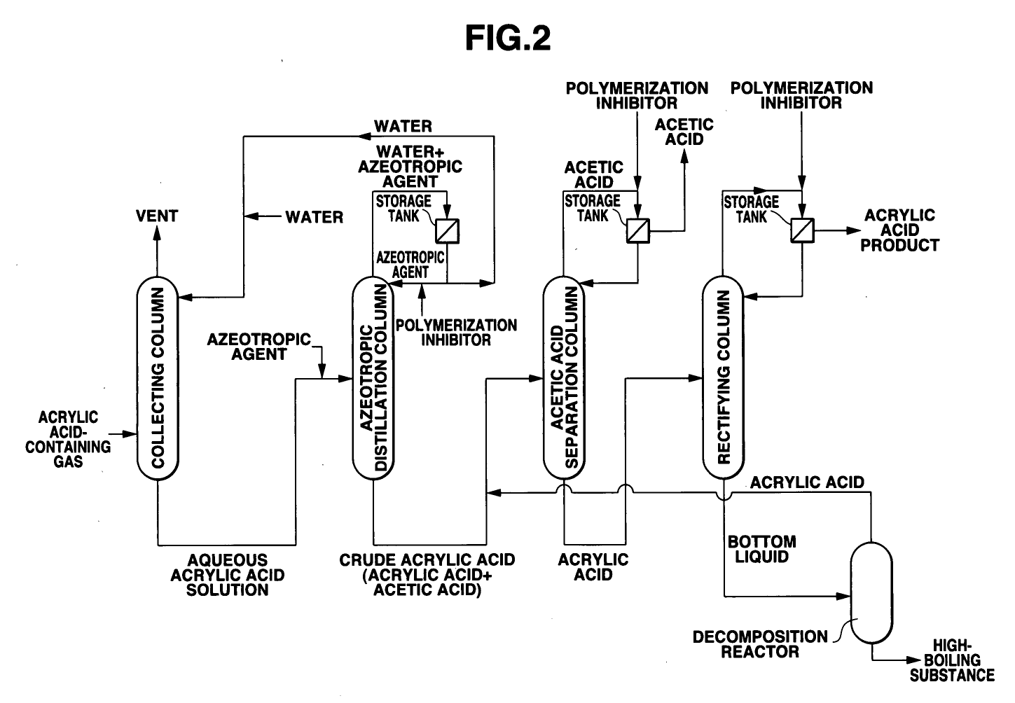

As shown in FIG. 2, an acrylic acid-containing gas obtained by subjecting propane, propylene and/or acrolein to gas-phase catalytic oxidation reaction using a molecular oxygen-containing gas, is introduced into an acrylic acid collecting column where the gas is contacted with water to obtain an aqueous acrylic acid solution.

Meanwhile, the above acrylic acid-containing gas may further contain N₂, CO₂, acetic acid, water, etc. Acetic acid, a part of water, N₂ and CO₂ are removed as a vent gas from a top of the collecting column.

The aqueous acrylic acid solution removed from the collecting column is fed together with an azeotropic agent to a distillation column, and an azeotropic mixture of water and the azeotropic agent is distilled off from a top of the distillation column, and crude acrylic acid containing acetic acid is obtained from a bottom thereof. The azeotropic mixture of water and the azeotropic agent which is distilled off from a top of the distillation column, is introduced into a storage tank to separate the mixture into an organic phase comprising mainly the azeotropic agent and a water phase comprising mainly water. The organic phase is mixed with a polymerization inhibitor, and then circulated to the distillation column. On the other hand, the water phase is circulated to the acrylic acid collecting column where the water phase is used as a collecting water to be contacted with the acrylic acid-containing gas. Meanwhile, if required, an additional amount of water may be replenished to the water-returning line. Also, in order to recover the azeotropic agent from the water circulated through the water-returning line, the water may be passed through an azeotropic agent recovery column (not shown) before circulating the water to the acrylic acid collecting column. Further, a part of the water may be discharged as a waste water out of the process.

The crude acrylic acid removed from the bottom of the distillation column, is introduced into an acetic acid separation column to separate residual acetic acid from the crude acrylic acid. The acetic acid is separated and removed from a top of the acetic acid separation column. Since the acetic acid removed from the top of the acetic acid separation column contains acrylic acid, a part of the acetic acid may be circulated to the process.

The acrylic acid obtained from the bottom of the acetic acid separation column contains substantially no acetic acid. The acrylic acid is introduced into a rectifying column to separate and remove high-boiling substances therefrom, thereby obtaining a high-purity acrylic acid product. A bottom liquid (high-boiling substances) obtained from the rectifying column is introduced into a high-boiling substance decomposition reactor (not shown).

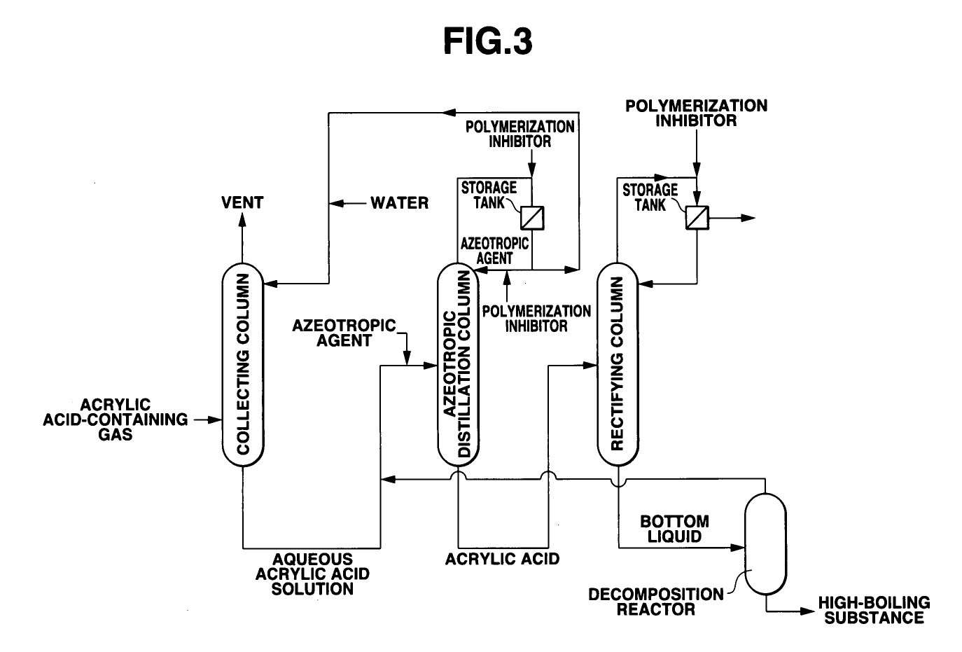

FIG. 3 is a flow sheet showing a process for production of acrylic acid using a distillation column having a combined function of dehydration and acetic acid separation in the process shown in FIG. 2.

The aqueous acrylic acid solution removed from the collecting column is mixed with an azeotropic agent, and then introduced into the distillation column. Water, acetic acid and the azeotropic agent are distilled off from the top of the distillation column. The azeotropic agent distilled is returned to the distillation column, and water and acetic acid distilled are returned to the collecting column. A part of water and acetic acid returned to the collecting column may be discharged as a vent gas out of the system. In addition, acetic acid and water which contain acrylic acid may be removed from a medium stage of the distillation column, and introduced into an acetic acid recovery column (not shown) to recover the acetic acid therefrom. A flow sheet for treatment of the bottom liquid obtained from the distillation column is the same as the flow sheet for treatment of the bottom liquid obtained from the acetic acid separation column as shown in FIG. 2.

On the other hand, methacrylic acid is produced by subjecting isobutylene or t-butyl alcohol as a starting material to the same oxidation and purification steps as described above.

Since methacrylic acid is an easily-polymerizable compound, it is well known that when subjecting the methacrylic acid to a purification step, especially a distillation step in which the methacrylic acid is heated and vaporized, polymers thereof tend to be produced. The thus produced polymers tend to be adhered onto an inner wall surface of the distillation column as well as packing materials or trays disposed therein, resulting in deteriorated quality of the treated products. In addition, the deposition of these polymers adhered tends to cause clogging of the distillation column (hereinafter referred to as “polymerization clogging”), so that the continuous operation of the distillation column tends to be no longer performed. In order to maintain a good quality of the treated products obtained in the distillation column and stably operate the distillation column, it is required to periodically overhaul the distillation column to remove the polymers adhered onto the inner wall surface, packing materials and trays. However, the overhaul procedure requires a huge labor and is time-consuming, resulting in significant deterioration in productivity.

Conventionally, to solve the problems due to production of the polymers, a polymerization inhibitor such as hydroquinone, p-methoxyphenol and phenothiazine has been added to the distillation column. Further, in Japanese Patent. Publication (KOKOKU) No. 52-34606 and Japanese Patent Application Laid-open (KOKAI) No. 2001-129388, there have been proposed the methods in which the polymerization inhibitor is added together with an oxygen gas to the distillation column.

In the method described in Japanese Patent Publication (KOKOKU) No. 52-34606, oxygen is introduced into the distillation column only from a bottom thereof such that a concentration of oxygen introduced is 0.01 to 5.0% by volume based on the acrylic acid vapor flow. Further, in Japanese Patent Application Laid-open (KOKAI) No. 2001-129388, it is described that oxygen may be fed to any of flow paths through which the fluid to be treated is flowed to the distillation column, and a concentration of the oxygen in the column is 0.1 to 1.0% by volume based on the acrylic acid vapor flow. However, the oxygen has been actually introduced into the distillation column only from the bottom thereof.

Thus, although various studies have been conventionally made on introduction of oxygen into the distillation column, any of these methods has failed to sufficiently prevent production of polymers within the distillation column, and has such a problem that the polymers produced are adhered and deposited in the column, resulting in clogging of the distillation column and failure of continuous operation thereof.

In view of the above conventional problems, an object of the present invention is to provide a process for stably conducting purification of (meth)acrylic acid by distillation for long period of time by preventing production of polymers of the (meth)acrylic acid and occurrence of the polymerization clogging in the distillation column.

### DISCLOSURE OF THE INVENTION

As a result of the present inventors' earnest study for solving the above problems, it has been found that when increasing an oxygen content in a liquid present within the distillation column or a fresh liquid formed by condensing a gas within the distillation column which is lacked due to insufficient feed for any reasons, an extremely high polymerization inhibiting effect can be obtained. The present invention has been attained on the basis of the above finding.

The reason why such a polymerization inhibiting effect can be obtained is considered as follows, though it is not clearly determined. That is, oxygen is required to prevent polymerization of (meth)acrylic acid. In order to effectively utilize the oxygen within the distillation column to prevent the polymerization, it is preferable that oxygen is dissolved in a liquid present within the distillation column, or a liquid freshly produced by condensing a gas within the distillation column. Therefore, it is effective to increase a solubility of oxygen in the liquid. For this reason, the oxygen partial pressure within the distillation column is preferably raised as highly as possible. However, since the distillation of (meth)acrylic acid as an easily-polymerizable substance is performed under reduced pressure to reduce the treating temperature for preventing the polymerization thereof, the increase in oxygen partial pressure within the distillation column requires the use of facilities having a large pressure-reducing capacity in order to treat the increased amount of gases within the distillation column and maintain the reduced pressure. Therefore, the method of increasing the oxygen partial pressure has not been actually applied to commercially available facilities.

Further, in the distillation column, since oxygen is continuously consumed, there is such a tendency that the oxygen concentration is high at a bottom thereof from which oxygen is conventionally introduced thereinto, and low at a top thereof. Also, within the distillation column maintained under reduced pressure, since the oxygen partial pressure itself is low, it takes a long time until the oxygen and the liquid within the distillation column reach equilibrium concentration therebetween, thereby failing to immediately obtain a sufficient oxygen concentration.

For these reasons, in the prior art, it is not possible to obtain a sufficient polymerization inhibiting effect even though oxygen is introduced into the distillation column only from the bottom thereof.

On the contrary, according to the present invention, since the dissolved oxygen concentration in the (meth)acrylic acid solution introduced into the distillation column is enhanced, it is possible to cause oxygen to directly act for preventing the polymerization of (meth)acrylic acid, thereby achieving a high polymerization inhibiting effect.

Further, the present inventors have found that the combination of specific polymerization inhibitors is especially effective for preventing the polymerization of (meth)acrylic acid.

Various aspects of the present invention are as follows:

1. A process for producing (meth)acrylic acid, comprising:

contacting a reaction gas containing (meth)acrylic acid obtained by gas-phase catalytic oxidation with an absorbent solvent to prepare a (meth)acrylic acid solution; and

introducing the (meth)acrylic acid solution into a distillation column to purify (meth)acrylic acid,

after adjusting a dissolved oxygen concentration in the (meth)acrylic acid solution to be introduced into the distillation column to not less than 12 ppm by weight, the (meth)acrylic acid solution being fed to the distillation column.

2. A process according to the above aspect 1, wherein the (meth)acrylic acid solution to be introduced into the distillation column is mixed with oxygen or an oxygen-containing gas to adjust the dissolved oxygen concentration in the (meth)acrylic acid solution.

3. A process according to the above aspect 2, wherein the (meth)acrylic acid solution to be introduced into the distillation column is mixed with oxygen or the oxygen-containing gas, and then introduced into the distillation column.

4. A process according to the above aspect 2, wherein the (meth)acrylic acid solution to be introduced into the distillation column is mixed with oxygen or the oxygen-containing gas, subjected to a gas-liquid separation, and then introduced into the distillation column.

5. A process according to any of the above aspects 2 to 4, wherein the mixing of the (meth)acrylic acid solution with oxygen or the oxygen-containing gas is performed in a conduit for introducing the (meth)acrylic acid solution into the distillation column, or a static mixer or an orifice disposed in the conduit.

6. A process according to the above aspect 1, wherein a means for the gas-liquid separation is a gas-liquid separation tank equipped with a pressure controlling apparatus.

7. A process according to the above aspect 1 or 2, wherein the dissolved oxygen concentration in the (meth)acrylic acid solution is adjusted in a facility disposed on an upstream side of the distillation column.

8. A process according to any of the above aspects 1 to 7, wherein the (meth)acrylic acid solution is in the form of an aqueous solution, the distillation column is an azeotropic dehydration distillation column, and at least a part of a phenol-based polymerization inhibitor is fed to the azeotropic dehydration distillation column from a raw material feed stage thereof or a position higher than the raw material feed stage, and a copper-based polymerization inhibitor is fed to the azeotropic dehydration column from a position lower than the raw material feed stage.

9. A process according to the above aspect 8, wherein the azeotropic dehydration column is any of a perforated plate column, a packed column and a combination of a perforated plate column and a packed column.

10. A process for producing (meth)acrylic acid, comprising:

subjecting propane, propylene, isobutylene or t-butanol to gas-phase catalytic oxidation;

contacting the obtained oxidation reaction mixture with water to prepare an aqueous (meth)acrylic acid solution; and

subjecting the aqueous (meth)acrylic acid solution to azeotropic dehydration distillation in the presence of an azeotropic agent,

upon the azeotropic dehydration distillation step, a phenol-based polymerization inhibitor being fed to an azeotropic dehydration distillation column from a position not lower than a raw material feed stage thereof, and

a copper-based polymerization inhibitor being fed to the azeotropic dehydration distillation column from a position lower than the raw material feed stage.

11. A process according to the above aspect 10, wherein the azeotropic dehydration distillation column is any of a perforated plate column, a packed column and a combination of a perforated plate column and a packed column.

12. A process according to the above aspect 10, wherein the phenol-based polymerization inhibitor is hydroquinone, methoquinone or a mixture thereof.

13. A process according to the above aspect 10, wherein the copper-based polymerization inhibitor is at least one material selected from the group consisting of copper dithiocarbamate, copper acetate, copper carbonate and copper acrylate.

The preferred process for producing acrylic acid according to the present invention is described in detail below.

Meanwhile, although the preferred embodiment in which the present invention is applied to production of acrylic acid is explained below, the present invention is not limited to the production of acrylic acid, and may also be applied to a process for production of methacrylic acid which includes the steps of contacting a reaction gas containing methacrylic acid obtained by subjecting isobutylene and/or t-butyl alcohol to gas-phase catalytic oxidation, with an absorbent solvent to prepare a methacrylic acid solution; and introducing the methacrylic acid solution into a distillation column to purify the (meth)acrylic acid solution by distillation.

First, the above aspects 1 to 9 of the present invention are described.

In the present invention, in the process for purifying acrylic acid by distillation as specifically shown in FIGS. 2 and 3, a sufficient amount of oxygen is dissolved in the acrylic acid solution to be introduced into the distillation column for preventing the polymerization thereof, and then introduced into the distillation column.

The acrylic acid solution, the azeotropic agent and polymerization inhibitor used in the distillation column, and oxygen or the oxygen-containing gas according to the present invention are explained below.

(1) Acrylic Acid Solution:

The acrylic acid solution to be treated by the present invention is not particularly restricted. The present invention may be most effectively applied to such a crude aqueous acrylic acid solution obtained by cooling a reaction gas produced by subjecting propane, propylene and/or acrolein to gas-phase catalytic oxidation using molecular oxygen, and/or absorbing the reaction gas in water. The crude acrylic acid aqueous solution obtained by the catalytic oxidation of propylene, etc., may contain, in addition to the aimed acrylic acid, by-products such as acetic acid, formic acid, formaldehyde and acetaldehyde.

(2) Distillation Column:

As the distillation column, there may be preferably used such a distillation column in which the number of theoretical plates is three or more. The upper limit of the number of theoretical plates within the distillation column is not particularly restricted, and is usually not more than 40 in view of costs for facilities used, etc., and more preferably 5 to 25. The type of the distillation column used in the present invention is not particularly restricted, and may be a plate column or a packed column. In the case of the plate column, about 10 to 80 trays may be usually used therein to provide the above number of theoretical plates.

As the preferable trays or packing materials used in the distillation column to which the process of the present invention is applied, there may be used those having a small differential pressure and a high efficiency as well as those having a simple structure with less projections from such a viewpoint that polymerizable substances are distilled therein. As the distillation column, there may be used a perforated plate column, a bubble-cap column, a packed column or a combination thereof (for example, combination of a perforated plate column and a packed column). Also, in the present invention, any of overflow weir, down corner, etc., may or may not be used in the distillation column without any limitation. Specific examples of the trays may include bubble-cap trays, perforated plate trays, bubble trays, super-flash trays, maxflux trays, dual trays or the like.

Examples of the packing material preferably used in the present invention may include conventional packing materials having various shapes such as a cylindrical shape, a hollow cylindrical shape, a saddle shape, a spherical shape, a cubic shape and a pyramidal shape as well as regular or irregular packing materials having specific shapes which are recently commercially available as high-performance packing materials.

Examples of these commercially available regular packing materials may include gauze-type regular packing materials such as “SULZER PACKING” produced by Sulzer Brothers Limited, “SUMITOMO SULZER PACKING” produced by Sumitomo Heavy Industries, Ltd., and “TECHNOPACK” produced by Mitsui & Co., Ltd.; sheet-type regular packing materials such as “MELLAPACK” produced by Sumitomo Heavy Industries, Ltd., “TECHNOPACK” produced by Mitsui & Co., Ltd., and “MC PACK” produced by Mitsubishi Chemical Engineering Corporation; grid-type regular packing materials such as “FLEXIGRID” produced by Koch Engineering Company, Inc.; as well as “GEMPAK” produced by Glitsch, Inc., “MONTZPACK” produced by Julius Montz. GmbH, “GOODROLL PACKING” produced by Tokyo Tokushu Kanaami, Inc., “HONEYCOMB PACK” produced by NGK Insulators. Ltd., “IMPULSE PACKING” produced by Nagaoka International Corporation, or the like.

Examples of the commercially available irregular packing materials may include RASCHIG RING, “PALL RING” produced by BASF AG, “CASCADE MINIRING” produced by Masstransfer Inc., “IMTP” produced by Norton Inc., “INTALOX SADDLE” produced by Norton Inc., “TELLERETTE” produced by Nittetsu Chemical Engineering Ltd., “FLEXIRING” produced by JGC Corporation, or the like.

The packing materials usable in the present invention are not limited only to the above described materials. In addition, the trays and packing materials may be used in combination, if required.

The pressure condition of the distillation column may be generally adjusted to a reduced pressure of about 2 to 40 kPa to reduce the operation temperature thereof. The bottom temperature of the distillation column is preferably kept at not more than 100° C.

(3) Azeotropic Agent and Polymerization Inhibitor:

In the process of the present invention, an organic solvent (azeotropic agent) capable of azeotropic distillation with water is used to efficiently conduct the dehydration distillation. Examples of the azeotropic agent usable in the present invention may include those capable of undergoing azeotropy with water and acetic acid, such as toluene, heptane, cyclohexane and isobutyl ether, and those incapable of undergoing azeotropy with acetic acid but capable of undergoing azeotropy with water, such as n-butyl acetate, isobutyl acetate, isopropyl acetate and methyl isobutyl ketone. These azeotropic agents may be used singly or in the form of a mixture of any two or more thereof. In the present invention, the kinds of azeotropic agents are not particularly restricted.

In general, the azeotropic agent acts as a diluent for acrylic acid. Therefore, a high concentration of the azeotropic agent inside the distillation column or in the bottom liquid thereof is preferable from the viewpoint of preventing the polymerization of acrylic acid. However, the concentration of the azeotropic agent may be determined so as to attain a well-balanced condition between the concentration and energy load required for the distillation.

Also, in the process of the present invention, in order to prevent the polymerization of acrylic acid, a polymerization inhibitor may be preferably added to at least one of a top of the distillation column, the bottom liquid and the acrylic acid solution to be introduced into the distillation column. The polymerization inhibitor used in the present invention is not particularly restricted, and various polymerization inhibitors described below may be suitably used. These polymerization inhibitors may be added in the form of a mixed solution with acrylic acid, azeotropic agent, water and/or a mixture thereof, and fed from the top, bottom and/or liquid feed stage of the distillation column.

(4) Oxygen or Oxygen-Containing Gas:

As the oxygen, there may be used an oxygen gas industrially produced.

The oxygen-containing gas contains a diluting gas for oxygen. As the diluting gas, there may be used at least one gas selected from nitrogen, carbon monoxide, carbon dioxide, water, argon and the like. The oxygen-containing gas preferably used in the present invention is air. Meanwhile, there may also be used the air diluted with nitrogen, etc., such that the oxygen concentration thereof is about 5 to 20% by volume.

As described above, the acrylic acid solution to be introduced into the distillation column is obtained by subjecting propylene and/or acrolein to gas-phase catalytic oxidation using molecular oxygen and then contacting the resultant reaction gas with water in the collecting column. In the collecting column, since oxygen is consumed by the gas-phase catalytic oxidation, the oxygen concentration therein is lower than that of air. For this reason, the acrylic acid solution removed from the bottom of the collecting column usually has a dissolved oxygen concentration of about 5 ppm which is as low as not more than 10% of a saturation solubility thereof.

Accordingly, in the present invention, in order to adjust the dissolved oxygen concentration of the acrylic acid solution having such a low dissolved oxygen concentration to not less than 12 ppm by weight, oxygen or the oxygen-containing gas is usually fed to and dissolved in the acrylic acid solution to enhance the dissolved oxygen concentration thereof.

The method of feeding oxygen gas or the oxygen-containing gas to the acrylic acid solution is not particularly restricted. For example, there may be used a method of disposing a feed nozzle for oxygen or the oxygen-containing gas in a conduit for introducing the acrylic acid solution into the distillation column and then blowing oxygen or the oxygen-containing gas into the conduit through the feed nozzle, a method of fitting a feed nozzle for oxygen or the oxygen-containing gas to a bottom portion of a facility disposed on an upstream side of the distillation column (in the acrylic acid production process, the acrylic acid-containing gas collecting column as shown in FIGS. 2 and 3 is generally used as the facility) and then blowing oxygen or the oxygen-containing gas into the bottom portion through the feed nozzle, or the like.

Further, in order to dissolve oxygen in the acrylic acid solution, there may be preferably provided an auxiliary device for the purpose of efficiently conducting gas-liquid contact between oxygen or the oxygen-containing gas and the acrylic acid solution. Among the auxiliary devices, as those disposed in the above conduit, there may be preferably used an orifice, a static mixer or the like, though not limited thereto. In the method of blowing oxygen or the oxygen-containing gas into the bottom portion of the facility disposed on an upstream side of the distillation column (for example, the acrylic acid-containing gas collecting column), as the auxiliary devices, there may be used a baffle plate in the form of a plain plate or a perforated plate, a gas sparger or the like, though not limited thereto.

Also, in the method of disposing a feed nozzle for oxygen or the oxygen-containing gas in a conduit for introducing the acrylic acid solution into the distillation column and then blowing oxygen or the oxygen-containing gas into the conduit through the feed nozzle, the thus fed oxygen or oxygen-containing gas may be fed together with the acrylic acid solution to the distillation column, or may be subjected to gas-liquid separation on an upstream side of the distillation column so as to prevent oxygen or the oxygen-containing gas from being supplied to the distillation column.

In the case where oxygen or the oxygen-containing gas added to the acrylic acid solution is separated therefrom by gas-liquid separation method, an appropriate gas-liquid separation facility may be disposed on an upstream side of the distillation column. The gas-liquid separation facility may be of any type capable of forming two gas and liquid phases, and various gas-liquid separation tanks may be preferably used for this purpose. The gas-liquid separation tanks may or may not be fitted with various equipments such as pressure control devices (valves) disposed in a discharge conduit connected to the tank, or a mist separator for preventing the liquid from being mixed in the gas.

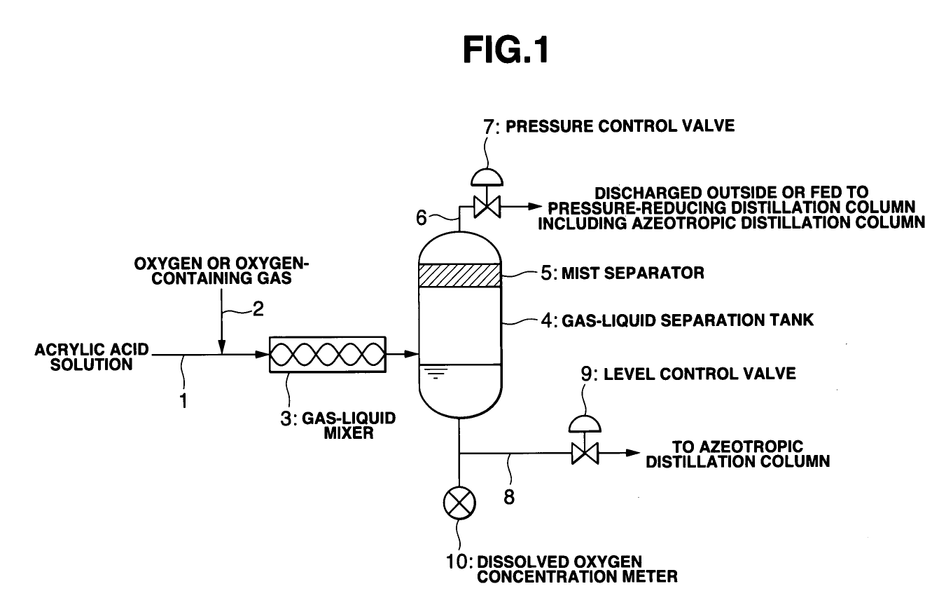

In FIG. 1, there is shown a dissolved oxygen concentration controlling means for conducting gas-liquid separation of the acrylic acid solution that is fed from the collecting column, distillation column, etc., to the next distillation column after mixing oxygen or the oxygen-containing gas therein. The acrylic acid solution flowed through conduit 1 is supplied with oxygen or the oxygen-containing gas through conduit 2, and both are then mixed together in gas-liquid mixer (static mixer) 3 and further fed to gas-liquid separation tank 4.

The gas-liquid separation tank 4 is provided therein with mist separator 5, and further connected at a top thereof to gas discharge conduit 6 equipped with pressure control valve 7 and at a bottom thereof to liquid discharge conduit 8 equipped with level control valve 9. Reference numeral 10 represents a dissolved oxygen concentration meter.

The acrylic acid solution that has been mixed with oxygen or the oxygen-containing gas in gas-liquid mixer 3 is subjected to gas-liquid separation in gas-liquid separation tank 4 to enhance the dissolved oxygen concentration therein, and then fed to the distillation column through conduit 8. The thus separated gas is removed through conduit 6 and then may be discharged as a waste gas after any treatments, if required, or may be fed to a pressure-reducing distillation column in the process.

Thus, in the present invention, the dissolved oxygen concentration in the acrylic acid solution to be introduced into the distillation column is controlled to not less than 12 ppm by weight by mixing oxygen or the oxygen-containing gas therein. When the dissolved oxygen concentration in the acrylic acid solution is not less than 12 ppm by weight, a sufficient polymerization inhibiting effect can be obtained. The upper limit of the dissolved oxygen concentration in the acrylic acid solution is not particularly restricted. Since a saturated dissolved oxygen concentration in the acrylic acid solution under 1 atm is 17 ppm by weight, the dissolved oxygen concentration in the acrylic acid solution is preferably controlled to 12 to 40 ppm by weight by using air of 1 to 3 atm in the consideration of facilitated procedure for mixing oxygen or the oxygen-containing gas (preferably air) in the acrylic acid solution, etc.

Thus, the polymerization of acrylic acid in the distillation column can be prevented by enhancing the dissolved oxygen concentration in the acrylic acid solution to be introduced into the distillation column. Therefore, in the present invention, it is not necessarily required to directly introduce oxygen or the oxygen-containing gas into the distillation column. However, oxygen or the oxygen-containing gas may be preferably fed to the bottom of the distillation column. The amount of oxygen or the oxygen-containing gas fed from the bottom of the distillation column may be preferably controlled such that the concentration of the oxygen-containing gas contained in a top gas of the distillation column is 0.01 to 0.2 mol %.

Next, the above aspects 10 to 13 of the present invention are explained.

In the present invention, a phenol-based polymerization inhibitor is fed to the azeotropic dehydration distillation column from a stage not lower than a raw material feed stage thereof, and a copper-based polymerization inhibitor is fed to the azeotropic dehydration column from a position lower than the raw material feed stage. Thus, by feeding the different polymerization inhibitors from the separate stages of the distillation column, it is possible to achieve a sufficient polymerization inhibiting effect even when the polymerization inhibitors are used in a economically small amount.

Examples of the phenol-based polymerization inhibitor may include hydroquinone, methoquinone (methoxy hydroquinone), cresol, phenol, t-butyl catechol or the like. Of these phenol-based polymerization inhibitors, preferred are hydroquinone, methoquinone or a mixture thereof. These phenol-based polymerization inhibitors may be used singly or in the form of a mixture of any two or more thereof.

The amount of the phenol-based polymerization inhibitor fed is usually 10 to 800 ppm by weight, preferably 50 to 600 ppm by weight based on the amount of acrylic acid fed to the distillation column. When the amount of the phenol-based polymerization inhibitor fed is too small, the polymerization inhibiting effect tends to be insufficient. On the contrary, when the amount of the phenol-based polymerization inhibitor fed is too large, although the polymerization inhibiting effect undergoes no adverse influence thereby, the use of more than necessary amount of the polymerization inhibitor is economically disadvantageous.

Examples of the copper-based polymerization inhibitor may include copper acetate, copper carbonate, copper acrylate, copper dithiocarbamates such as copper dimethyldithiocarbamate, copper diethyldithiocarbamate, copper dipropyldithiocarbamate, copper dibutyldithiocarbamate, copper dipentyldithiocarbamate, copper dihexyldithiocarbamate, copper diisopropyldithiocarbamate, copper diisobutyldithiocarbamate, copper methylisopropyldithiocarbamate, copper piperidiyldithiocarbamate, copper morpholinyldithiocarbamate and copper diphenyldithiocarbamate, or the like. Of these copper-based polymerization inhibitors, preferred is at least one of copper dibutyldithiocarbamate, copper acetate, copper carbonate and copper acrylate. These copper-based polymerization inhibitors may be used singly or in the form of a mixture of any two or more thereof.

The amount of the copper-based polymerization inhibitor fed is usually 1 to 100 ppm by weight, preferably 10 to 80 ppm by weight based on the amount of acrylic acid fed to the distillation column. When the amount of the copper-based polymerization inhibitor fed is too small, the polymerization inhibiting effect tends to be insufficient. On the contrary, when the amount of the copper-based polymerization inhibitor fed is too large, the use of more than necessary amount of the polymerization inhibitor is economically disadvantageous, and further the bottom portion of the distillation column tends to be corroded.

Further, the above polymerization inhibitors may be used, if required, in combination with oxygen gas ordinarily used as polymerization inhibitor as well as other polymerization inhibitors. Examples of the other polymerization inhibitors may include phenothiazine compounds such as phenothiazine, bis-(α-methylbenzyl) phenothiazine, 3,7-dioctyl phenothiazine and bis-(α,α′-dimethylbenzyl)phenothiazine; N-oxyl compounds such as tert-butyl nitroxide, 2,2,6,6-tetramethyl-4-hydroxypiperidyl-1-oxyl, 2,2,6,6-tetramethylpiperidyl-1-oxyl, 2,2,6,6-tetramethylpiperidinooxyl, 4-hydroxy-2,2,6,6-tetramethylpiperidinooxyl and 4,4′,4″-tris-(2,2,6,6-tetramethylpiperidinooxyl)phosphite; phenylenediamines such as p-phenylenediamine; nitroso compounds such as N-nitrosodiphenylamine; ureas such as urea; thioureas such as thiourea; or the like. In the case where the phenol-based polymerization inhibitor is used in the form of a mixture with the other polymerization inhibitors (except for the copper-based polymerization inhibitors), the amount of the phenol-based polymerization inhibitor used (or the total amount of the two or more phenol-based polymerization inhibitors) is usually not less than 30% by weight, preferably not less than 60% by weight. Also, in the case where the copper-based polymerization inhibitor is used in the form of a mixture with the other polymerization inhibitors, the amount of the copper-based polymerization inhibitor used (or the total amount of the two or more copper-based polymerization inhibitors) is usually not less than 1% by weight, preferably not less than 10% by weight.

The above phenol-based or copper-based polymerization inhibitor is kept in a liquid or solid state at an ordinary temperature and, therefore, can be directly fed to a desired stage of the distillation column. However, since these polymerization inhibitors can sufficiently prevent the polymerization of acrylic monomers even when used in a small amount, the polymerization inhibitors are preferably used in the form of a solution or slurry in a solvent from the standpoints of uniform feeding as well as saving of costs. The phenol-based polymerization inhibitor may be fed to the azeotropic dehydration distillation column from the raw material feed stage thereof. In this case, the phenol-based polymerization inhibitor may be preferably dissolved in the raw material.

As the above solvent, there may be used water or organic solvents. Examples of the organic solvents may include ketones such as acetone, methyl ethyl ketone and methyl isobutyl ketone; carboxylic acids such as acetic acid, propionic acid, acrylic acid and methacrylic acid; aromatic hydrocarbons such as benzene, toluene and xylene; esters such as methyl acetate and butyl acetate; or the like. These solvents may be used singly or in the form of a mixture of any two or more thereof. Of these mixtures of solvents, preferred are a mixture of water and toluene, a mixture of water and acrylic acid, and crude acrylic acid containing dimers and trimers of acrylic acid.

The distillation procedure of the present invention may be conducted by either continuous distillation or batch distillation. The distillation conditions are not particularly restricted, and may be determined according to kinds and contents of impurities contained in the acrylic monomers.

The temperature of the bottom liquid discharged from the azeotropic dehydration distillation column is preferably not more than 100° C. Since the azeotropic dehydration distillation is usually conducted under reduced pressure, the temperature of the bottom liquid discharged from the azeotropic dehydration distillation column may be controlled by adjusting the vacuum degree at the top of the distillation column. The pressure at the top of the azeotropic dehydration distillation column is usually controlled to 13.3 to 39.9 kPa (100 to 300 mmHg).

### PREFERRED EMBODIMENT OF THE PRESENT INVENTION

The present invention is described in more detail by the following Experimental Example, Examples and Comparative Examples. Meanwhile, all of the “%” and “ppm” used herein represent “% by weight” and “ppm by weight”, respectively.

### Experimental Example 1

Aqueous acrylic acid solutions at 25° C. having different concentrations from each other were prepared, and then tested to measure a saturation solubility of oxygen therein under an oxygen atmosphere of 1 atm using a dissolved oxygen concentration meter. The results are shown in Table 1.

TABLE 1

| Aqueous acrylic acid solution | | Saturation |
|---|---|---|
| Acrylic acid | | solubility of |
| concentration | Water concentration | dissolved oxygen |
| (wt. %) | (wt. %) | (wt. ppm) |
| 0 | 100 | 40 |
| 40 | 60 | 61 |
| 60 | 40 | 88 |
| 80 | 20 | 149 |
| 100 | 0 | 323 |

(Examples Corresponding the Above Aspect 1 to 9 of the Present Invention)

### Example 1

An aqueous acrylic acid solution as a raw liquid to be distilled which was obtained from an acrylic acid-containing gas collecting column as shown in FIG. 2 and contained 55% by mass of acrylic acid, 1.5% by mass of acetic acid, 0.3% by mass of formaldehyde and a slight amount of formic acid, was introduced into an azeotropic distillation column (the number of theoretical plates: 9) to conduct azeotropic dehydration distillation of acrylic acid. In this case, toluene was used as an azeotropic agent.

Upon initiating the operation of the azeotropic distillation column, distillation was conducted using toluene to stabilize an inside of the azeotropic distillation column. Then, the above aqueous acrylic acid solution prior to feeding to the azeotropic distillation column was mixed with diluted oxygen to adjust a dissolved oxygen concentration thereof to 20 ppm, and then fed to a 16th stage tray of the azeotropic distillation column at a feed rate of 1,100 kg/hr. Meanwhile, the dissolved oxygen concentration in the raw liquid before mixing with oxygen was 7 ppm, and the saturation solubility of oxygen in the raw liquid was about 85 ppm.

The diluted oxygen mixed with the aqueous acrylic acid solution was separated from the aqueous acrylic acid solution before introducing the solution to the azeotropic distillation column. Further, toluene was fed to a 30th stage tray of the azeotropic distillation column at a feed rate of 3,100 kg/hr. In addition, air that was diluted 3 times with a nitrogen gas was fed from the bottom of the azeotropic distillation column such that the oxygen concentration in a top gas of the azeotropic distillation column was 0.05 mol %. While controlling the top pressure to 14.0 kPa, hydroquinone and phenothiazine as polymerization inhibitors were fed to the top of the azeotropic distillation column in such an amount that the concentrations of the respective polymerization inhibitors in the bottom liquid were 800 ppm for hydroquinone and 500 ppm for phenothiazine. At this time, the bottom temperature of the azeotropic distillation column was 83° C., and the top temperature thereof was 41° C.

As a result, it was confirmed that even after continuing the operation of the azeotropic distillation column for 3 months, no increase in differential pressure inside the azeotropic distillation column was observed, and polymerization clogging was effectively prevented.

### Example 2

The same procedure as defined in Example 1 was conducted except that air was used instead of the diluted oxygen, and mixed in the aqueous acrylic acid solution prior to feeding to the azeotropic distillation column to adjust the dissolved oxygen concentration thereof to 15 ppm.

As a result, it was confirmed that even after continuing the operation of the azeotropic distillation column for 3 months, no increase in differential pressure inside the azeotropic distillation column was observed, and polymerization clogging was effectively prevented.

### Example 3

The same procedure as defined in Example 2 was conducted except that the aqueous acrylic acid solution in which air was mixed was directly fed to the azeotropic distillation column without separating the air therefrom.

As a result, it was confirmed that even after continuing the operation of the azeotropic distillation column for 3 months, no increase in differential pressure inside the azeotropic distillation column was observed, and polymerization clogging was effectively prevented.

### Comparative Example 1

The same procedure as defined in Example 1 was conducted except that the aqueous acrylic acid solution as the raw liquid was directly fed to the azeotropic distillation column without adjusting the dissolved oxygen concentration thereof.

As a result, it was confirmed that after continuing the operation of the azeotropic distillation column for 3 months, a differential pressure inside the azeotropic distillation column was increased by 2.8 kPa.

### Example 4

A packed column equipped with a 1,000 ml glass flask at a bottom thereof, a distillation pipe at a top thereof and a raw material feed pipe at a mid portion thereof was used to conduct azeotropic distillation of an aqueous acrylic acid solution. The raw feed material was prepared from crude acrylic acid obtained by gas-phase catalytic oxidation reaction of propylene, and was composed of 51.5% of acrylic acid, 2.5% of acetic acid and 46.0% of water.

Hydroquinone and methoquinone as phenol-based polymerization inhibitors were added in an amount of 200 ppm for each, to the above aqueous acrylic acid solution. The aqueous acrylic acid solution was fed to the distillation column at a feed rate of 275 g/hr. Further, an acrylic acid solution containing copper dibutyldithiocarbamate (in an amount corresponding to 60 ppm based on acrylic acid as the raw material) was fed from the bottom of the packing material filled in the distillation column at the position corresponding to the first-stage theoretical plate thereof at a feed rate of 10 g/hr. The distillation procedure was conducted while circulating the azeotropic agent composed of toluene as a refluxing liquid. In addition, air was fed to the distillation column from the bottom thereof through a capillary tube at a feed rate of 5 ml/min. The operation conditions are shown below in Table 2.

TABLE 2

| | |
|---|---|
| Bottom temperature | 90° C. |
| Top temperature | 50° C. |
| Top pressure | 23.94 kPa (180 Torr) |

As a result of gas chromatographic analysis of the liquid removed from the bottom of the distillation column under a steady operation condition thereof, it was confirmed that the bottom liquid was composed of 89.7% of acrylic acid, 3.7% of acetic acid, 0.3% of water and 6.3% of toluene. Further, when continuing the operation of the distillation column for 10 hours, it was confirmed that no production of polymers inside the distillation column and in the bottom liquid was recognized.

### Examples 5 and 6 and Comparative Examples 2 to 4

The same procedure as defined in Example 4 was conducted except that the kind and adding position of polymerization inhibitor were changed variously, thereby conducting the azeotropic dehydration distillation. The results are shown in Tables 3 and 4. The continuous distillation time for evaluating the operation of the distillation column was 10 hours similarly to that of Example 4. Meanwhile, in Table 4, it is described that as to the results of Comparative Examples, the continuous distillation operation was “stopped” with the elapse of less than 10 hours. This means that the 10-hour continuous distillation operation was impossible since the difference in pressure between the top and bottom of the distillation column reached not less than 1.33 kPa (10 Torr) due to clogging inside the distillation column which was caused by production of acrylic polymers.

TABLE 3

| | Example 4 | Example 5 | Example 6 |
|---|---|---|---|
| Phenol-based | Hydroquinone | Hydroquinone | Hydroquinone |
| inhibitor | (200 ppm) | (200 ppm) | (200 ppm) |
| | Methoquinone | Methoquinone | Methoquinone |
| | (200 ppm) | (200 ppm) | (200 ppm) |
| | | | Phenothiazine |
| | | | (200 ppm) |
| Feeding | Raw material | Refluxing | Raw material |
| position of | feed stage | stage | feed stage |
| phenol-based | | | |
| inhibitor | | | |
| Copper-based | Copper | Copper | Copper |
| inhibitor | dibutyldithio- | dibutyldithio- | dibutyldithio- |
| | carbamate | carbamate | carbamate |
| | (60 ppm) | (60 ppm) | (60 ppm) |
| Feeding | First-stage | First-stage | First-stage |
| position of | theoretical | theoretical | theoretical |
| copper-based | plate | plate | plate |
| inhibitor | | | |
| Results | Distillation | Distillation | Distillation |
| | for 10 hours | for 10 hours | for 10 hours |
| | No turbidity | No turbidity | No turbidity |
| | observed at | observed at | observed at |
| | bottom | bottom | bottom |

TABLE 4

| | Comparative | Comparative | Comparative |
|---|---|---|---|
| | Example 2 | Example 3 | Example 4 |
| Phenol-based | Hydroquinone | Methoquinone | — |
| inhibitor | (200 ppm) | (200 ppm) | |
| | Methoquinone | | |
| | (200 ppm) | | |
| Feeding | Raw material | Raw material | — |
| position of | feed stage | feed stage | |
| phenol-based | | | |
| inhibitor | | | |
| Copper-based | — | Copper | Copper |
| inhibitor | | dibutyldithio- | dibutyldithio- |
| | | carbamate | carbamate |
| | | (60 ppm) | (60 ppm) |
| Feeding | — | Raw material | First-stage |
| position of | | feed stage | theoretical |
| copper-based | | | plate |
| inhibitor | | | |
| Results | Stopped after | Stopped after | Stopped after |
| | 8 hours | 5 hours | 2 hours |
| | White | No turbidity | No turbidity |
| | turbidity | observed at | observed at |
| | observed at | bottom | bottom |
| | bottom | | |

### INDUSTRIAL APPLICABILITY

According to the present invention, there is provided a process for stably producing (meth)acrylic acid for a long period of time by effectively preventing production of polymers of the (meth)acrylic acid and further polymerization clogging.

1.-9. (canceled)

10. A process for producing (meth)acrylic acid, comprising:
subjecting propane, propylene, isobutylene or t-butanol to gas-phase catalytic oxidation;
contacting the obtained oxidation reaction mixture with water to prepare an aqueous (meth)acrylic acid solution; and
subjecting the aqueous (meth)acrylic acid solution to azeotropic dehydration distillation in the presence of an azeotropic agent,
upon the azeotropic dehydration distillation, a phenol-based polymerization inhibitor being fed to an azeotropic dehydration distillation column from a position not lower than a raw material feed stage thereof, and
a copper-based polymerization inhibitor being fed to the azeotropic dehydration distillation column from a position lower than the raw material feed stage.

11. A process according to claim 10, wherein the azeotropic dehydration distillation column is any of a perforated plate column, a packed column and a combination of a perforated plate column and a packed column.

12. A process according to claim 10, wherein the phenol-based polymerization inhibitor is hydroquinone, methoquinone or a mixture thereof.

13. A process according to claim 10, wherein the copper-based polymerization inhibitor is at least one material selected from the group consisting of copper dithiocarbamate, copper acetate, copper carbonate and copper acrylate.
